(19) United States
(12) Patent Application Publication
(10) Pub. No.: US 2004/0186044 A1
(43) Pub. Date: Sep. 23, 2004

(54) Modulation of myelination by interaction with P75 and TRK receptors

(76) Inventors: Jose Miguel Cosgaya, Stanford, CA (US); Jonah R. Chan, Cupertino, CA (US); Eric M. Shooter, Stanford, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/700,980

(22) Filed: Nov. 3, 2003

Related U.S. Application Data
(60) Provisional application No. 60/424,430, filed on Nov. 6, 2002.

(51) Int. Cl. G01N 33/53; G01N 33/567

(52) U.S. Cl. 514/2; 435/7.2

(57) ABSTRACT

Methods are provided for the treatment of demyelinating disease in the central or peripheral nervous system. Myelination is enhanced by administration of agents that are agonists of the p75^{NTR} receptor; and/or that inhibit a Trk receptor, including TrkC receptor. Methods of screening for pharmaceutically active compounds that enhance myelination are also provided.

The myelin sheath is a unique component of the nervous system that functions to maximize the efficiency and velocity of action potentials transmitted through nerve cell axons. The composition of myelin differs in the peripheral and central nervous systems principally in the nature of the proteins that are embedded in the lipid bilayers of the multiple myelin wraps. The proteins in myelin have received considerable attention because of their role in demyelinating diseases.

The formation of myelin is a complex, dynamic process. The myelin forming glia and the neurons are involved in a series of neuronal-glial interactions controlling the various stages of myelination. These include the proliferation and migration of glial cells on axons in the proliferative stage, the elongation and ensheathment of the axon in the premyelination stage, and the initiation, rate, and extent of growth of the myelin sheath in the final myelination stage.

The neurotrophins are a small family of dimeric secretory proteins that affect essentially all biological aspects of vertebrate neurons, including their survival, shape and function. In mammals, the known neurotrophins are nerve growth factor (NGF), brain-derived neurotrophic factor (BDNF), neurotrophin-3 (NT3) and neurotrophin-4 (NT4). These proteins share many functional properties with classical neurotransmitters; for example, they are released at synapses and they are required for activity-dependent forms of synaptic plasticity.

Neurotrophins have long been known to promote the survival and differentiation of vertebrate neurons. However, these growth factors can also induce cell death through the p75 neurotrophin receptor (p75^{NTR}), a member of the tumor necrosis factor receptor superfamily. Consistent with a function in controlling the survival and process formation of neurons, p75^{NTR} is mainly expressed during early neuronal development, and in the adult, p75^{NTR} is re-expressed in various pathological conditions, including epilepsy, axotomy and neurodegeneration.

The broad spectrum of biological activities exerted by the neurotrophins results from their ability to bind and activate two structurally unrelated receptor types, the p75^{NTR} and the three members (in mammals) of the Trk receptor family of tyrosine kinases. p75^{NTR} is one of roughly 25 members of the TNF receptor superfamily and binds all neurotrophins with similar nanomolar affinities.

It has been proposed that neurotrophins promote neuronal survival by activating Trk receptors, and they cause cell death by activating p75NTR. Both p75^{NTR} and Trk receptors are frequently coexpressed in the same neurons, raising questions as to the mechanisms triggering pro-or anti-apoptotic cascades after neurotrophin binding.

Methods of enhancing myelination of neurons damaged by inflammation, injury and the like, are of considerable clinical interest. The present invention addresses this issue.

Relevant Literature

U.S. Pat. No. 5,468,872; and International Application WO/9507911, describe the use of K-252a functional derivatives to potentiate neurotrophin-3 for the treatment of neurological disorders. U.S. Pat. No. 6,225,282 describes the treatment of hearing impairments with a trkB or trkC agonist. Human trk receptors and neurotrophic factor inhibitors are described in U.S. Pat. Nos. 6,027,927; 5,910,574; and 5,877,016. Modulators of TrK activity are disclosed in International Patent application WO0203071. Antibodies that mimic the action of neurotrophins are discussed in International Application WO9515180. Antibodies specific for p75 are disclosed in WO/9706251.

The biology of neurotrophins and their receptors are reviewed, for example, by Dechant and Barde (2002) Nature Neuroscience 5:1131-1136; and Dechant (2001) Cell Tissue Res. 305:229-238.

### SUMMARY OF THE INVENTION

Methods are provided for the treatment of demyelinating disease in the central or peripheral nervous system. Myelination is enhanced by administration of agents that are agonists of the p75^{NTR} receptor; and/or that inhibit a Trk receptor, including TrkC receptor. It is found that activation of the p75NTR receptor increases myelination, and activation of Trk receptor(s) decreases myelination. Methods of screening for pharmaceutically active compounds that enhance myelination are also provided.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Expression profiles for MAG, P0, BDNF, and NT3 during the myelination process in Schwann cell_neuronal cocultures. (A) Western blot analysis of MAG and P0 in Schwann cell/neuronal cocultures throughout the myelination process. (B) ELISA for BDNF and NT3 in cultures of Schwann cells and DRG neurons alone, and in cocultures. Schwann cells were induced to myelinate at ˜7 days after seeding onto DRG neurons (day 0) with the addition of ascorbic acid. The results are shown as the mean value ±SD.

FIG. 2. Western blot analysis of the effects of endogenous and exogenous neurotrophins on the expression of myelin proteins in Schwann cell/neuronal cocultures. Exogenous BDNF (100 ng_ml), TrkB-Fc (1 μg/ml), NT3 (100 ng/ml), and TrkC-Fc (1 μg/ml) were added to cocultures on the day of induction of myelin formation. After 6 days of induction, cocultures were extracted and probed for MAG and P0 proteins. The results are shown as the mean value ±SD.

FIG. 3. The effects of endogenous and exogenous neurotrophins in the formation of myelin internodes in Schwann cell/neuronal cocultures determined by immunocytochemical analysis of P0. (A) Control cultures without the addition of primary antibody. (B) Control cultures with the addition of the anti-P0 antibody. (C) Addition of exogenous BDNF (100 ng/ml). (D) Addition of TrkB-Fc (1 μg/ml). (E) Addition of exogenous NT3 (100 ng/ml). (F) Addition of TrkC-Fc (1 μg/ml). All factors were added to cocultures on the day of induction of myelin formation. Immunocytochemistry was performed on cocultures after 6 days of induction.

FIG. 4. Western blot analysis of the effect of neurotrophins and neurotrophin scavengers during the development of the sciatic nerve. Newborn mice (P1) were s.c. injected with 3 μg each of BDNF, NT3, TrkB-Fc, or TrkC-Fc as indicated under Example 1. The contralateral leg was injected with vehicle alone as a control for each one of the individually treated mice. Two days later (P3, 2 days treatment), the sciatic nerves were isolated and processed for Western blot analysis. In some instances, the animals were reinjected at this stage and allowed to proceed for 2 more days (P5, 4 days treatment). The sciatic nerves were then processed and analyzed in the same manner. (A) Quantification of MAGprotein content after treatment with the different factors for 2 or 4 days. Representative Western blots are shown (Lower). (B) Quantification of P0 protein content and representative Western blots. The results are shown as the mean value ±SD of the percentage of the levels expressed in the contralateral leg (injected with saline vehicle alone).

FIG. 5. Effects of BDNF treatment on the myelin ultrastructure during sciatic nerve development. Newborn mice (P1) were injected with 3 μg of BDNF as indicated in Example 1 and reinjected again 2 days later. The contralateral leg served as a control with the injection of saline vehicle alone. At P5 the sciatic nerves from both legs were removed and processed for the electron microscopy study. Low magnification electron micrographs from (A) control nerve treated with saline alone and (B) BDNF-treated nerve. Axons ensheathed by Schwann cell cytoplasm without the formation of myelin are indicated with an asterisk. The scale bar represents 1 μm. (C) Ensheathed and myelinated axons from control and BDNF-treated nerves were counted and the proportions shown as a percentage of the sum of both. (D) The thickness of the myelin sheath was determined by counting the number of lamellae of individual myelinated axons. The distribution is shown as the mean value ±SD of the percentage of myelinated axons that falls within a certain range in the number of lamellae.

FIG. 6. Effects of TrkB-Fc treatment on the myelin ultrastructure during sciatic nerve development. Newborn mice were injected with 3 μg of TrkB-Fc and their sciatic nerves analyzed as in FIG. 5. Electron microphotographs from (A) control nerve (saline alone) and (B) TrkB-Fc-treated nerve. (C) Distribution of myelinated axons against ensheathed axons as a percentage of the sum of both. (D) The thickness of the myelin sheath was determined by counting the number of lamellae. Myelinated axons were distributed as a function of the thickness of the myelin sheath. The distribution is shown as the mean value ±SD of the percentage of myelinated axons that falls within the given range in the number of lamellae.

FIG. 7. Schematic model representing the modulation of the endogenous levels of neurotrophins and their possible roles during myelin formation. BDNF and NT3 are expressed at high levels during the initial phases of myelin development. Concomitant with the proliferation and premyelination phases, there is a marked decrease in NT3 levels, whereas BDNF remains constant. High levels of NT3 do not allow the myelination program to proceed further and keep the Schwann cell/axonal unit in an ensheathed premyelinated stage. When NT3 levels are diminished, the Schwann cell initiates the formation of a myelin internode surrounding the axon. On the contrary, high levels of BDNF are required for the myelination process to proceed and BDNF levels will decrease only after the myelination program has already been initiated. Elevated levels of BDNF during the early stages of myelination increase the speed and extent of the final process. An illustration of the timeline for the proliferation, premyelination, and myelination stages of Schwann cells in the coculture system appears below the model.

FIG. 8: p75^{NTR,} TrkB and TrkC are present during development in sciatic nerve and SC/DRG cocultures. (A) Expression of neurotrophin receptors, the myelin protein PMP22 and the ribosomal protein L19 were analyzed by RT-PCR from purified rat DRG, SC, premyelinating SC/DRG cocultures before induction of myelination (SC/DRG day 0), actively myelinating cocultures after 4 days of induction (SC/DRG day 4), newborn mouse sciatic nerve, and adult mouse brain. (B) Protein levels of the myelin protein P₀ and the neurotrophin receptors p75^{NTR,} TrkC-TK+ and TrkB-T1 were analyzed at the times indicated by Western-blot in SC/DRG cocultures and rat sciatic nerve. The results are presented as mean value ±SD.

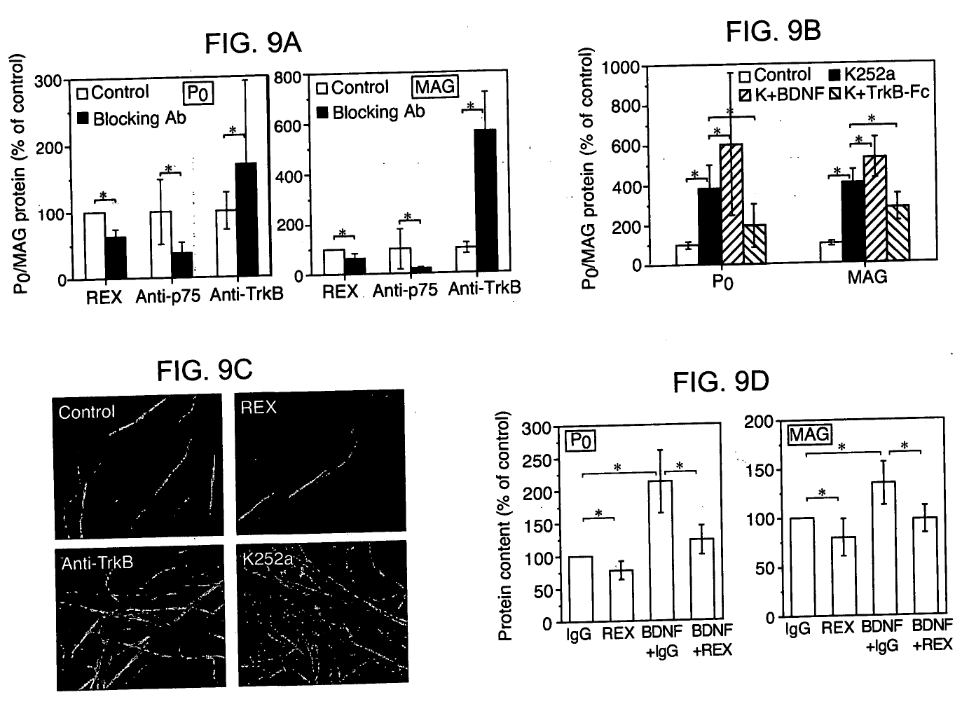

FIG. 9: p75^{NTR} and Trk receptors have opposite effects on myelination. Rat SC/DRG cocultures were treated for 6 days with (A) blocking antibodies against p75^{NTR} (REX and anti-p75) or TrkB antiserum (anti-TrkB), or with (B) the Trk tyrosine kinase inhibitor K252a in combination with BDNF or TrkB-Fcat the time of induction. P₀ and MAG content was determined by Western-blot analysis. Asterisks: A): P<0.01; B): P<0.05. (C) Mature myelin internodes were visualized by immunocytochemistry using an antibody against P₀. Cocultures were maintained in the presence or absence of REX, anti-TrkB or K252a. (D) p75^{NTR} blocking antibodies inhibited myelin expression in vivo. Newborn mice were injected with REX (n=7), BDNF+IgG (n=5) or BDNF+REX (n=6). Four days later the sciatic nerves were isolated and myelin protein expression was analyzed by Western-blot. Asterisks: P<0.05. (E) Injection of REX decreases the thickness of myelin sheaths from sciatic nerve axons. Newborn mice were injected with REX and the sciatic nerves were extracted and processed for electron microcopy. Representative electron micrographs as well as the myelin thickness distribution from IgG- and REX-treated nerves are shown. The difference in the distribution of the number of lamellae between IgG- and REX-treated samples (21.4±10.0 and 16.4±7.3 lamellae, respectively) is statistically significant (p<0.0001). (F) Thick myelin sheaths are absent in sciatic nerve axons from p75^{NTR} −/− mice. Sciatic nerves from 5 days old wild-type and p75^{NTR} −/− littermate mice were extracted and myelin thickness was analyzed by electron microscopy as before. The distributions of the number of lamellae from wild-type and p75^{NTR} −/− mice (20.4±10.4 and 17.9±6.8 lamellae, respectively) are statistically different (p<0.0001).

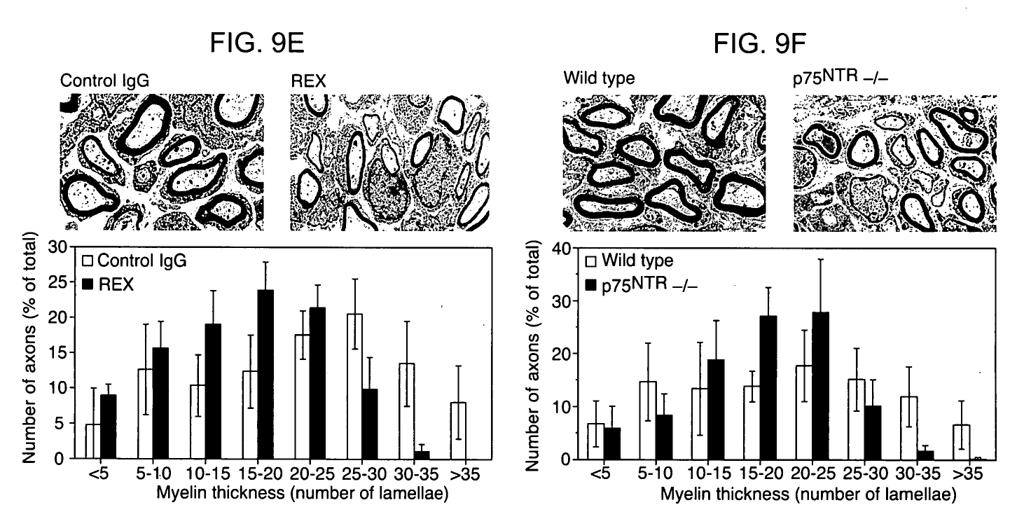

FIG. 10: The modulation of endogenous BDNF levels does not affect the myelination process in the p75^{NTR} −/− mice. (A-B) Newborn wild-type and p75^{NTR} −/− mice were injected with BDNF (wild-type, n=10; p75^{NTR} −/−, n=25) and NT3 (wild-type, n=13; p75^{NTR} −/−, n=14) or the scavengers TrkB-Fc (wild-type, n=13; p75^{NTR} −/−, n=20) and TrkC-Fc (wild-type, n=14; p75^{NTR} −/−, n=20) and the levels of (A) P₀ and (B) MAG were analyzed by Western-blot 4 days later. Asterisks: p<0.01. (C-D) Wild-type and p75^{NTR} −/− SC/DRG cocultures were established from E13 mouse embryos obtained through crossing heterozygous (p75^{NTR} +/−) mice. Levels of (C) P₀ and (D) MAG were determined by Western-blot six days after initiation of myelination in the presence of the different factors. Asterisks: p<0.01.

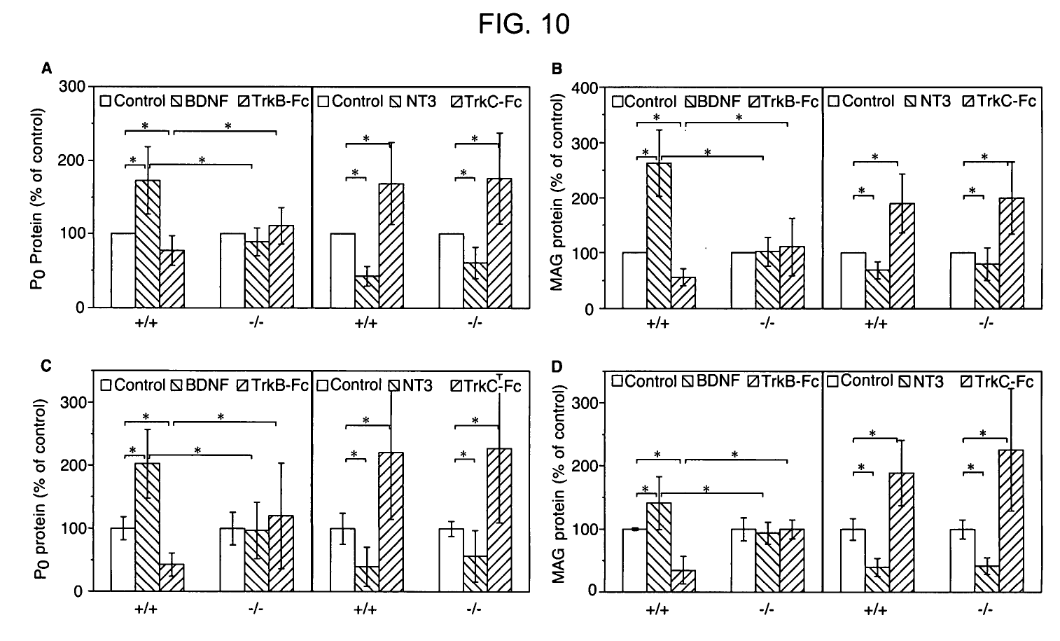

FIG. 11: Actions of endogenous neurotrophins and their receptors throughout myelination. During glial proliferation, elongation and ensheathment, NT3 levels decrease while TrkC and p75^{NTR} remain constant. The activation of TrkC by NT3 during these phases prevents the myelination program from proceeding. When myelination is initiated NT3 protein levels have already become undetectable, thereby removing its inhibitory action. At the same time, BDNF acts as a positive modulator of myelination through the activation of p75^{NTR}. Once active myelination is underway, extracellular BDNF is removed through its binding to the increased levels of TrkB-T1. After myelination is complete, all the neurotrophins and their receptors are down-regulated.

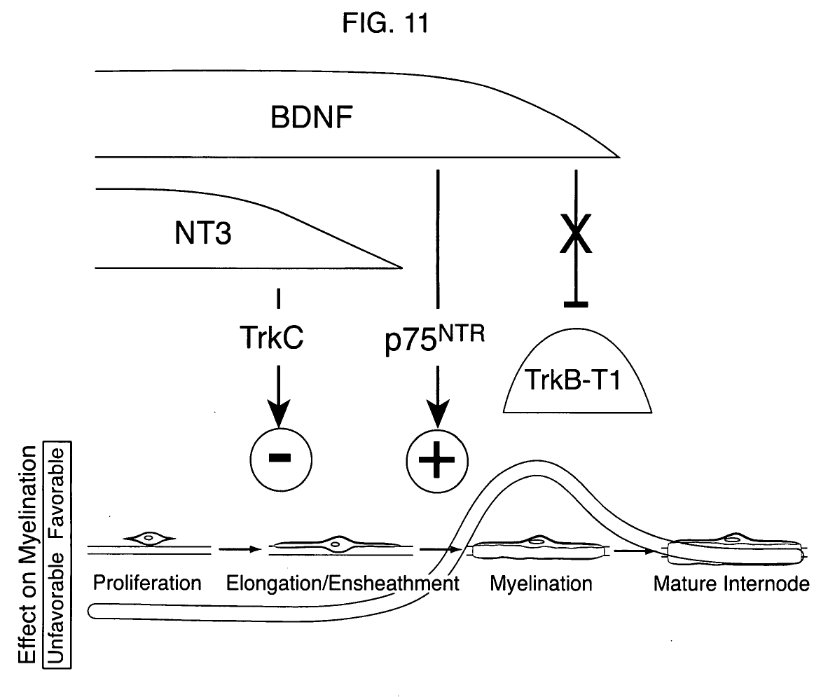

### DESCRIPTION OF THE SPECIFIC EMBODIMENTS

Methods are provided for the treatment of demyelinating disease by administration of one or both of (a) an agonist of the p75NTR receptor and (b) an antagonist or inhibitor of the Trk receptor. The p75NTR receptor and the Trk receptor(s) have been found to have opposite effects on myelination, such that activation of p75^{NTR} increases myelination, while activation of Trk decreases myelination. Myelination is therefore enhanced by inhibiting or blocking Trk receptors, or by binding to or activating p75^{NTR}.

The therapeutic agent may be administered before, during or after the onset of disease or injury. To induce myelination of peripheral neurons, a compound from either or both class of therapeutic agent may be administered. To induce myelination of central nervous system neurons, it may be preferable to administer agonists or inhibitors of Trk. Methods of screening for therapeutic compounds are also provided, for example by determining the binding or activation of a neuronal receptor selected from the group of p75^{NTR}, TrkA, TrkB, TrkC. Screening methods may detect, for example, the kinase activity of a Trk receptor; or one of the known biological activities of these receptors. Initial screening assays may detect binding of a candidate agent to the receptor. Of particular interest is the development of agents that act specifically on the targeted receptor.

As used herein, the term “treating” is used to refer to both prevention of disease, and treatment of pre-existing conditions. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

An effective dose is the dose that, when administered for a suitable period of time, usually at least about one week, and may be about two weeks, or more, up to a period of about 4 weeks, 8 weeks, or longer will evidence an increase in the myelination of targeted cells. It will be understood by those of skill in the art that an initial dose may be administered for such periods of time, followed by maintenance doses, which, in some cases, will be at a reduced dosage.

The compounds can be incorporated into a variety of formulations for therapeutic administration. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation.

For some conditions, particularly central nervous system conditions, it may be necessary to formulate agents to cross the blood brain barrier (BBB). One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic or imaging compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g., intravascular (intravenous, intraarterial, intracapillary) administration, injection into the cerebrospinal fluid, intracavity or direct injection. Intrathecal administration maybe carried out through the use of an Ommaya reservoir, in accordance with known techniques. (F. Balis et al., Am J. Pediatr. Hematol. Oncol. 11, 74, 76 (1989).

The effective amount of a therapeutic composition to be given to a particular patient will depend on a variety of factors, several of which will be different from patient to patient. A competent clinician will be able to determine an effective amount of a therapeutic agent to administer to a patient to enhance myelination. Utilizing LD₅₀ animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

The methods of the invention are of interest for the treatment of demyelinating conditions, which may be acute, e.g. from an injury, or chronic, e.g. relating to genetic or autoimmune disease. Conditions of interest include multiple sclerosis, EAE, optic neuritis, acute transverse myelitis, acute disseminated encephalitis, Guillain-Barre syndrome, Marie-Charcot-Tooth disease, and injuries resulting in neuronal grow and remyelination. The targeted cells may be in the central nervous system, or in the peripheral nervous system.

Multiple Sclerosis (MS) is the most common central nervous system (CNS) demyelinating disease, affecting 350,000 (0.1%) individuals in North America and 1.1 million worldwide. Attacks of neurologic impairment occur in the early phase, which is characterized histologically by inflammatory lesions containing a predominance of CD4 T cells, B cells and both MHC class II positive macrophages and microglia, a resident CNS antigen presenting cell (APC). After multiple acute attacks a chronic “secondary progressive” phase with sustained neurologic impairment often ensues. This “irreversible” phase is characterized by neuronal loss and atrophy.

Clinical symptoms of MS include sensory loss (paresthesias), motor (muscle cramping secondary to spasticity) and autonomic (bladder, bowel, sexual dysfunction) spinal cord symptoms; cerebellar symptoms (eg, Charcot triad of dysarthria, ataxia, tremor); fatigue and dizziness; impairment in information processing on neuropsychological testing; eye symptoms, including diplopia on lateral gaze; trigeminal neuralgia; and optic neuritis. The subject therapy may be used in conjunction with anti-inflammatory agents, or immunomodulatory agents.

Charcot-Marie-Tooth disease (CMT), also named hereditary motor and sensory neuropathies, includes a clinically and genetically heterogeneous group of disorders affecting the peripheral nervous system. Traditionally, the different classes of CMT have been divided into demyelinating forms (CMT1, CMT3, and CMT4) and axonal forms (CMT2), a clinically very useful distinction. However, investigations of the underlying molecular and cellular disease mechanisms, mainly accomplished using cell culture and animal models, as well as specific re-examination of appropriate patient cohorts, have revealed that the pathological signs of myelinopathies and axonopathies are often intermingled. These findings reflect the dependence and intimate cellular interactions of Schwann cells and neurons, mainly during nerve development and, as indicated by the pathology of CMT, also in the adult organism. The methods of the invention may be used in the treatment of demyelinating forms of the disease.

Guillain-Barre Syndrome, also called acute inflammatory demyelinating polyneuropathy and Landry's ascending paralysis, is an inflammatory disorder of the peripheral nerves—those outside the brain and spinal cord. It is characterized by the rapid onset of weakness and, often, paralysis of the legs, arms, breathing muscles and face. GBS is the most common cause of rapidly acquired paralysis in the United States today, affecting one to two people in every 100,000. It typically begins with weakness and/or abnormal sensations of the legs and arms. It can also affect muscles of the chest, face and eyes. Although many cases are mild, some patients are virtually paralyzed. Breathing muscles may be so weakened that a machine is required to keep the patient alive. Many patients require an intensive care unit during the early course of their illness, especially if support of breathing with a machine is required. Although most people recover, the length of the illness is unpredictable and often months of hospital care are required. The methods of the invention find use in treating GBS, in combination with supportive therapy, rehabilitation, and the like.

Mammalian species that may be treated with the present methods include canines and felines; equines; bovines; ovines; etc. and primates, particularly humans. Animal models, particularly small mammals, e.g. murine, lagomorpha, etc. may be used for experimental investigations. Other uses include investigations where it is desirable to investigate a specific effect in the absence of T cell mediated inflammation.

The methods of the present invention also find use in combined therapies. For example, the FDA has approved for MS the long-term use of beta-interferons and glatiramer acetate, which is a synthetic form of myelin basic protein (MBP) that has fewer side effects than interferon. The combined use of, for example, immunomodulatory agents and myelinating enhancers can have the advantages that the required dosages for the individual drugs is lower, and the effect of the different drugs complementary.

Known inhibitors of Trk receptors include the molecule K-252a, and derivatives thereof, for example as described by U.S. Pat. No. 5,468,872; and International Application WO/9507911, herein incorporated by reference. The Trk receptors are tyrosine kinases, and as such may be inhibited by inhibitors of this class of enzyme, or by molecules that compete for the binding site of ligands, e.g. BDNF. p75^{NTR} is a member of the TNF receptor superfamily, and comprises a “death domain”. Agonists of p75^{NTR} may bind to and activate the site of ligand binding on the receptor.

In screening assays for biologically active agents, cells, usually cells expressing at least one Trk or p75^{NTR} receptor, are contacted with the agent of interest, and the effect of the agent assessed by monitoring output parameters, such as expression of markers, cell viability, and the known responses to activation of the receptor, e.g. autophosphorylation of Trk receptors, and the like. The cells may be freshly isolated, cultured, genetically altered as described above, or the like, including, for example, fibroblasts expressing exogenous Trk receptors, which cells are known to proliferate in response to neurotrophins.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

Alternatively, binding studies may be performed in cell-free systems, using methods known in the art for determining the specific binding of a receptor and candidate agent.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected markers. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81).

It is to be understood that this invention is not limited to the particular methodology, protocols, formulations and reagents described, as such may, of course, vary. It is also to be understood that the terminology used herein is for the purpose of describing particular embodiments only, and is not intended to limit the scope of the present invention, which will be limited only by the appended claims.

It must be noted that as used herein and in the appended claims, the singular forms “a”, “and”, and “the” include plural referents unless the context clearly dictates otherwise. Thus, for example, reference to “a complex” includes a plurality of such complexes and reference to “the formulation” includes reference to one or more formulations and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the methods and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, and pressure is at or near atmospheric.

### EXPERIMENTAL

### EXAMPLE 1

Endogenous brain-derived neurotrophic factor (BDNF) and neurotrophin-3 (NT3) are identified in Schwann cell dorsal root ganglia/neuronal cocultures, and are shown to modulate the myelination program of the peripheral nervous system. The differential expression of BDNF and NT3 were examined and compared with the expression profiles of myelin proteins in the cocultures throughout the myelination process. B DNF levels correlated with active myelin formation, whereas NT3 expression was initially high and then down regulated throughout the proliferation and premyelination periods. Addition of exogenous BDNF enhanced myelination, whereas the removal of the endogenous BDNF by using the BDNF receptor TrkB-Fc fusion protein inhibited the formation of mature myelin internodes. Interestingly, exogenous NT3 significantly inhibited myelination, whereas the removal of the endogenous NT3 by using the NT3 receptor TrkC-Fc fusion protein resulted in an enhancement similar to that obtained with the addition of BDNF. In addition, in vivo studies were performed during the development of the mouse sciatic nerve. Subcutaneous injections of BDNF resulted in an enhancement of myelin formation in the sciatic nerve, whereas the removal of the endogenous BDNF dramatically inhibited myelination. Injections of NT3 inhibited myelin formation, and the removal of the endogenous NT3 enhanced myelination. These results demonstrate that BDNF and NT3 possess different modulatory roles in the myelination program of the peripheral nervous system and that their mechanisms of action are specific and highly regulated.

Our results demonstrate that endogenous BDNF and NT3 have different expression profiles during the development of myelin in these cultures and that they exert different modulatory actions on the myelination program in the peripheral nervous system (PNS). We have confirmed these effects in vivo during PNS myelin development, providing one more example of the complex and dynamic regulation of Schwann cell/neuronal interactions, this time by neurotrophins.

Materials and Methods

Materials. NGF was obtained from Serotec. BDNF and NT3 were gifts from Regeneron Pharmaceuticals (Tarrytown, N.Y.). TrkB-Fc and TrkC-Fc were also gifts from Regeneron Pharmaceuticals and are chimeric proteins of the Fc fraction of the human Ig fused to the extracellular domain of TrkB or TrkC receptors, respectively.

Dorsal Root Ganglia (DRG) Neuronal Schwann Cell Cocultures. Purified neuronal and Schwann cell cultures were prepared as previously described. Neuronal cultures were established from DRG neurons obtained from Sprague-Dawley rat embryos at 15 days gestation (Simonsen Laboratories, Gilroy, Calif.). DRG neurons were dissociated and plated onto collagen coated coverslips. Nonneuronal cells were eliminated by cycling (three 2-day cycles) with a fluorodeoxyuridine-containing medium (10 μM). NGF-dependent neurons were then maintained for 1 week in a medium consisting of 10% FBS in MEM and 100 ng/ml of NGF.

Schwann cells were isolated from the sciatic nerve of 4-day-old rat pups. Schwann cells were purified by using cytosine arabinoside and Thy-1.1 antibody mediated lysis of the fibroblasts (the anti-Thy-1.1 antibody was obtained from the American Type Culture Collection). Approximately 100,000 purified Schwann cells were than seeded onto purified neuronal cultures of ˜50,000 cells and allowed to proliferate and ensheath the axons (˜1 week). Myelination was then initiated with the addition of ascorbic acid (50 μg/ml), which was replenished with feeding every 2 to 3 days.

Western Blot Analysis. Samples from Schwann cell/neuronal co-cultures and sciatic nerves were prepared for Western blot analysis by homogenization in radioimmunoprecipitation assay (RIPA) buffer [PBS with 1% Nonidet P-40/0.5% deoxycholate/0.1% SDS/1 mM PMSF/Complete protease inhibitor tablets (Roche Molecular Biochemicals)] followed by high-speed centrifugation. Protein determination was made by using the Bicinchoninic Acid Kit (Sigma). Equivalent amounts of total protein extract from each sample were mixed with sample buffer, boiled, and loaded onto SDS polyacrylamide gels. Electrophoretic separation of the extracts was typically performed on 10-15% (depending on the molecular weight of the protein of interest) discontinuous acrylamide gels under denaturing conditions. The proteins were then transferred to pure nitrocellulose membranes (PROTRAN BA85, Schleicher and Schuell, 0.45 μm) and probed with specific antibodies. The mouse monoclonal anti-myelin-associated glycoprotein (MAG; Chemicon) was used at a concentration of 2.5 μg/ml (under nonreducing conditions); the mouse monoclonal anti-P0 antibody was used at a dilution of 1:5,000. All primary antibodies were incubated overnight at 4° C. Secondary HRP-conjugated anti-mouse IgG and anti-rabbit IgG antibodies were used at a dilution of 1:10,000 (Jackson ImmunoResearch). The blots were developed by chemiluminescence (Renaissance, DuPont_NEN) as described by the manufacturer. All of the blots were imaged and quantitated in the linear range for the corresponding antibodies. Protein concentrations of the samples were serial diluted until linear intensities were achieved. In many instances, the blots were stripped and reprobed with different antibodies.

Immunocytochemistry. Cocultures were fixed in 4% paraformaldehyde before dehydration through a graded ethanol series (50, 70, 90, and 100%). Samples were then permeabilized and blocked by incubation with 20% normal goat serum or 10% FCS. Primary antibodies included the mouse monoclonal anti-P0 antibody used at a dilution of 1:500 and the mouse monoclonal anti-MAG at a concentration of 2.5 μg/ml. The Texas red-conjugated anti-mouse IgG (Jackson ImmunoResearch) was used as a secondary antibody at a dilution of 1:1,000. Cellular nuclei were examined by using the Hoechst dye. Samples were mounted and fluorescence microscopy was accomplished by using a Nikon Microphot FXA.

ELISA Analysis. ELISAs were performed by using the TMB (tetramethylbenzidine) Peroxidase Substrate System (Kirkegaard & Perry Laboratories). Briefly, the BDNF and NT3 ELISAs were accomplished by using Immobilon plates (Nunc) coated with the TrkB-Fc fusion protein and the #4704 anti-NT3 antibody (Regeneron Pharmaceuticals), respectively, followed by incubation of the samples. The BDNF ELISA was developed by using a polyclonal chicken anti-BDNF anti-serum and an anti-chicken-HRP antibody (Promega) for detection. The NT3 ELISA was developed by using a biotinylated monoclonal anti-NT3 antibody (Regeneron Pharmaceuticals) and streptavidin-HRP (Sigma) for detection. The substrate was incubated in the ELISA reaction for ˜2-5 min or until adequate signal was detected. ELISA reactions were stopped with the addition of 1 M phosphoric acid. Using a Bio-Rad Model 550 Microplate Reader, optical densities were measured at a wavelength of 450 nm.

Injections in Mouse Sciatic Nerve. BDNF, NT3, TrkB-Fc, and TrkC-Fc (3 μg each; Regeneron Pharmaceuticals) were injected s.c. starting from the caudal portion of the greater trochanter region and running parallel along the sciatic nerve (total volume of 5 μl). T he contralateral leg served as a control for each factor with the injection of saline. Injections were performed on 1-day-old mouse pups (C57BU6, Simonsen Laboratories) and the sciatic nerves were extracted and processed 48 h later. A second set of mice were reinjected with the factors and then examined after an additional 48 h (4 days of total treatment). Nerves used for electron microscopy were trimmed and incisions were made at the flexure of the greater trochanter. In total, 11 animals were analyzed after injection with BDNF, 12 with TrkB-Fc, 11 with NT3, and 7 with TrkC-Fc.

Electron Microscopy. Electron microscopy was performed by the Electron Microscopy Facility in the Department of Microbiology and Immunology (Stanford University, CA). Processing of the sciatic nerve was accomplished by fixation in 2% glutaraldehyde and 4% paraformaldehyde solution in PBS, followed by post-fixation in 1% OSO₄. Staining was achieved with 1% aqueous uranyl acetate, followed by dehydration with an ethanol gradient and treatment with propylene oxide. Finally, samples were infiltrated and embedded in pure epoxy.

Results

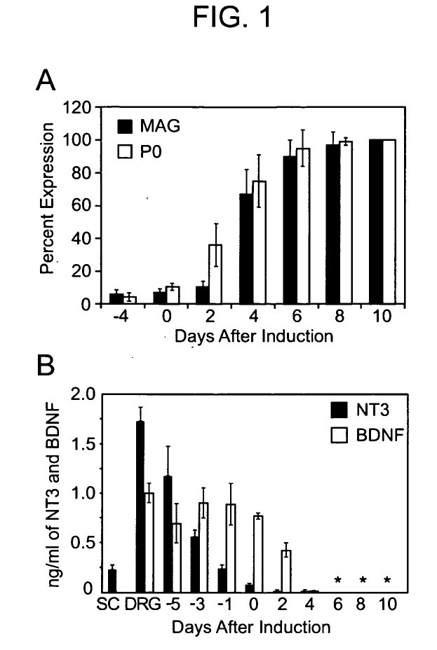

Expression Profiles of Endogenous Neurotrophins During Myelination in Schwann Cell_Neuronal Cocultures. The synthesis of neurotrophins and the expression of neurotrophin receptors have previously been documented in Schwann cells and DRG neurons. To investigate the role of neurotrophins in the myelination process, BDNF and NT3 levels were examined in Schwann cell/neuronal cocultures established as described in Materials and Methods. The cells were grown to maturity separately and contaminating cells were removed. Approximately 1 week after removal of the antimitotic agent, neuronal cultures were seeded with Schwann cells. On contact with the axons, Schwann cells proliferated rapidly (proliferation stage). Approximately 4 days after seeding, the axons were fully populated and proliferation had ceased. The Schwann cells then began to elongate and ensheath the axons (premyelination stage). At this time (7 days after seeding the Schwann cells) the cocultures were induced to myelinate by the addition of ascorbic acid (myelination stage). Active myelin formation occurred between ˜4 and 7 days after induction, as characterized by lipid analyses, internode diameter measurements, and electron microscopy. By examining the expression profiles of MAG and P0 in the Schwann cell_neuronal cocultures (FIG. 1A), the initial induction of the myelin protein synthesis was observed ˜2 days after the addition of ascorbic acid. The expression of MAG and P0 leveled off by 6 days after induction, suggesting that active myelin synthesis was complete at this time. This extensive and reproducible characterization of the cocultures allowed for further detailed investigations and/or distinctions to be made between the proliferation, premyelination, and myelination phases of the Schwann cells as noted above.

Because the DRG neurons were consistently cultured in the presence of NGF, only the expression of BDNF and NT3 were examined. Conditioned media from neurons, Schwann cells, or cocultures were collected every two days and assayed for BDNF and NT3 by ELISA (FIG. 1B). Whereas DRG neurons secreted both BDNF and NT3 at high concentrations (1-2 ng/ml), Schwann cells only secreted NT3, at concentrations 10-fold lower than from the DRG neurons (0.2 ng/ml). On seeding the Schwann cells onto the DRG neurons, NT3 levels immediately decreased. In addition, during the proliferation and premyelination periods, NT3 levels gradually diminished until near undetectable amounts were observed at the day of induction. Interestingly, BDNF levels remained relatively constant throughout the entire proliferation and premyelination periods, and only began to decrease at 2 days after induction of the onset of active myelin synthesis (FIG. 1B). Both BDNF and NT3 were undetectable at about 6 days after induction with ascorbic acid. These results demonstrate different expression profiles for BDNF and NT3 throughout the myelination process in Schwann cell/neuronal cocultures, which implies a potential difference in function.

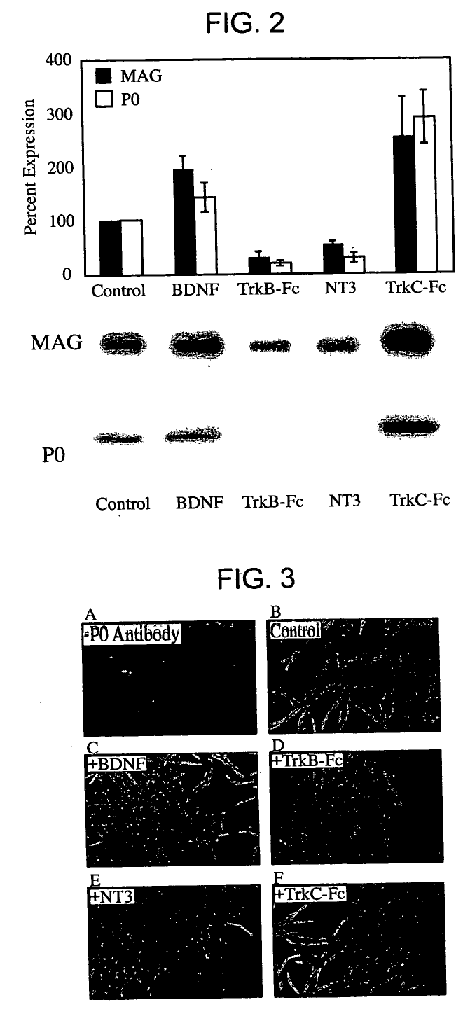

Endogenous BDNF and NT3 Exert Different Modulatory Actions on Myelination in Schwann Cell_Neuronal Cocultures. To investigate the potential role of BDNF and NT3 on the myelination process, exogenous BDNF (100 ng/ml) or NT3 (100 ng/ml) were added at the day of induction in Schwann cell/neuronal cocultures. BDNF significantly enhanced the expression of MAG by ˜2-fold and P0 by ˜1.5-fold over control cultures, whereas NT3 diminished the expression of these myelin proteins by ˜2- and 3-fold, respectively (FIG. 2). To further examine the role of endogenous BDNF and NT3, the TrkB-Fc and TrkC-Fc fusion proteins were used to diminish the endogenous neurotrophin levels. The addition of TrkB-Fc (1 μg/ml) at the day of induction inhibited the expression of MAG by 3-fold and P0 by 5-fold, whereas the addition of TrkC-Fc (1 μg/ml) resulted in an enhancement greater than that obtained with the addition of BDNF (˜3-fold; FIG. 2). These results indicate that endogenous BDNF plays an important role in signaling the initiation of myelin formation, whereas endogenous NT3 acts as an inhibitor of the myelination process.

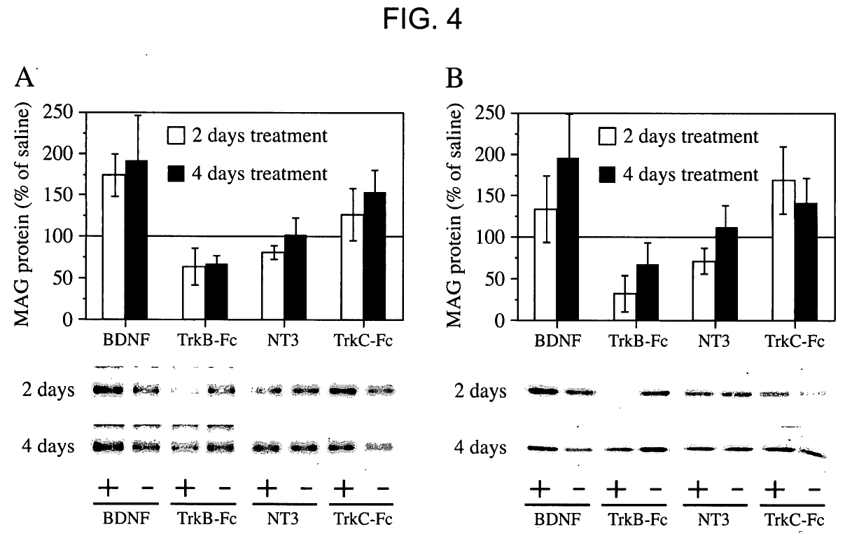

To examine the effects of BDNF and NT3 on the formation of mature myelin internodes, immunocytochemical analyses for P0 were performed in the presence of exogenous neurotrophins or the BDNF and NT3 scavengers TrkB-Fc and TrkC-Fc. Six days after the addition of ascorbic acid to the Schwann cell/neuronal cocultures, the formation of mature myelin internodes was detected through P0 immunostaining (FIG. 3B). The addition of BDNF produced an enhancement in myelin formation, especially at earlier time points, although this effect was not easily distinguishable through fluorescence microscopy at 6 days after induction because of the high degree of myelination in control conditions (FIG. 3C). In contrast, the removal of endogenous BDNF by addition of TrkB-Fc greatly inhibited the formation of mature myelin internodes (FIG. 3D). Conversely, NT3 had the opposite effect on myelin formation. Exogenous NT3 inhibited the appearance of myelin internodes, whereas TrkC-Fc, by removing NT3, enhanced myelin formation (FIGS. 3E and F). As was noted with the addition of BDNF, the enhancement by TrkC-Fc was significantly greater at earlier time points, but was not easily distinguishable at 6 days after induction. In addition, the few internodes that were detected after addition of TrkB-Fc or NT3 were significantly thinner and shorter in length than internodes obtained from the control cultures. Neither BDNF nor NT3 caused any changes in Schwann cell proliferation or in the morphology of the ensheathed Schwann cells. There were no observable changes in the axonal processes of the DRG neurons and there was no sign of increased cell death, as determined by neurofilament and nuclear staining. These results suggest that under our in vitro culture conditions, endogenous BDNF and NT3 modulate the myelination process in opposite ways. BDNF is required for proper myelin internode formation, whereas an excess of NT3 inhibits the myelination process and the formation of normal myelin internodes.

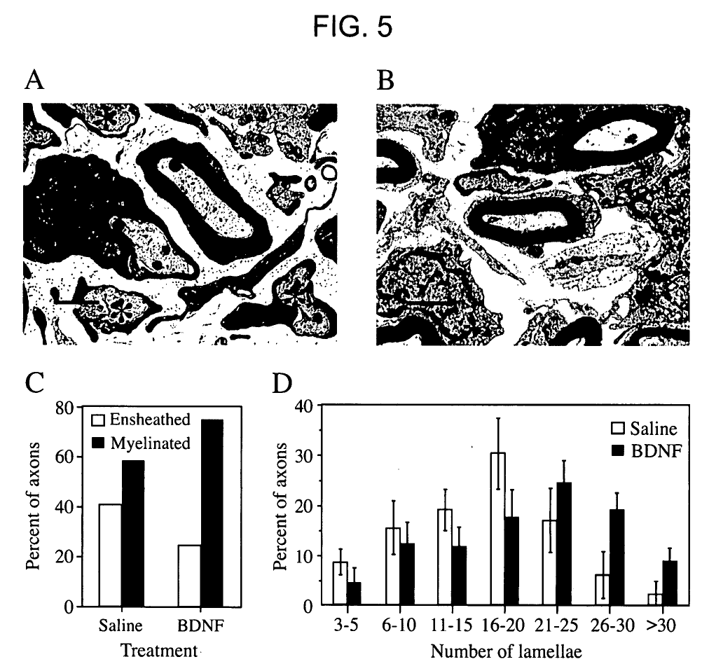

Endogenous BDNF and NT3 Exert Different Modulatory Actions on Myelination in the Developing Sciatic Nerve. The effects of BDNF and NT3 on myelin formation in vivo were analyzed during the development of the sciatic nerve in newborn mice. Subcutaneous injections of the neurotrophins or the neurotrophin scavengers were made starting at the caudal portion of the greater trochanter region and running parallel along the sciatic nerve. Injections of BDNF, NT3, TrkB-Fc, and TrkC-Fc (3 μg each) were performed on 1-day-old mouse pups, followed by the extraction and processing of the sciatic nerves for Western blot analyses 48 h later. A second set of mice were reinjected with the same factors and then examined after an additional 48 h (4 days of total treatment). Contralateral legs were injected with an equivalent volume of the saline buffer used as vehicle and provided the specific controls for the injections of all of the factors reported. BDNF was found to significantly enhance the expression of MAG and P0 in the sciatic nerves by more than 50% (FIG. 4). On the contrary, injections of NT3 inhibited MAG and P0 expression in the sciatic nerves of mice treated for 2 days by ˜25%.

Interestingly, the inhibition by NT3 was time-dependent and was not detected in mice treated for a total of 4 days. In agreement with the results from the coculture experiments, the physiological effects of BDNF and NT3 in mature myelin formation were clearly demonstrated after injection with the neurotrophin scavengers. TrkB-Fc reduced both P0 and MAG levels, whereas TrkC-Fc significantly enhanced the expression of the myelin proteins in both the 2- and 4-day-treated animals. The effects obtained with both of the receptor-fusion proteins demonstrate once again that endogenous BDNF and NT3 possess different modulatory actions in vivo, during the development of the sciatic nerve.

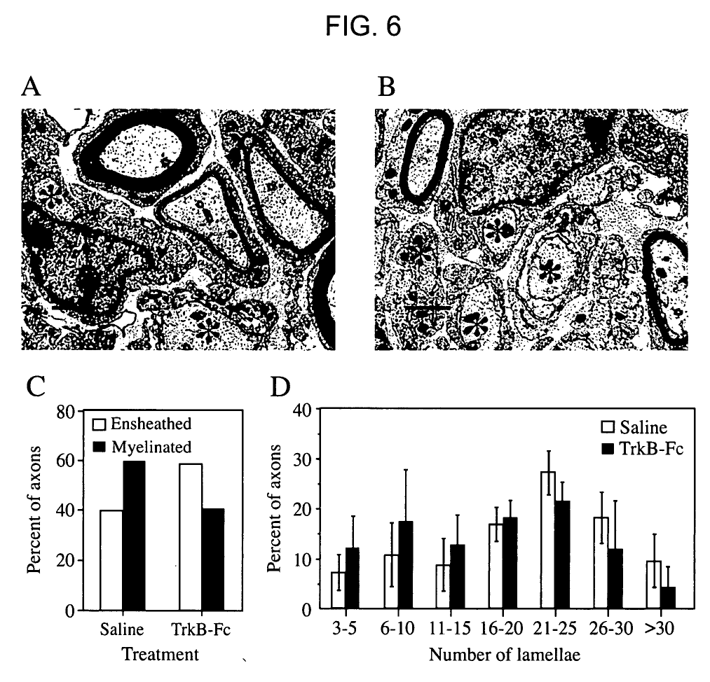

To analyze the effect of BDNF on the formation of the myelin sheath in the sciatic nerves, electron microscopy was used (FIGS. 5A and B). Sciatic nerves treated with BDNF displayed a 2-fold decrease in ensheathed axons accompanied by a significant correlative increase in the number of myelinated axons (FIG. 5C). BDNF not only produced an increase in the number of myelinated axons, but also an enlargement of the myelin sheath itself. By examining the distribution of the number of lamellae in the myelinated axons of the control nerves and the BDNF-treated nerves, it became evident that the myelin sheath of the BDNF nerves were significantly thicker. This increase in size was due to an increase in the number of lamellae in the myelin internodes. FIG. 5D shows the distribution of the thickness (number of wraps) of the myelinated axons from control and BDNF-treated nerves. Whereas the control nerves had a higher percentage of axons with lower number of wraps of myelin, the BDNF-treated nerves had a profile that was shifted, because of a greater percentage of axons with a higher number of wraps. Less than 10% of the myelinated axons in the control nerve had more than 25 wraps of myelin, whereas more than 30% of all of the myelinated axons in the BDNF-treated nerves-were in this same population. On average, BDNF-treated nerves had 25% more wraps of myelin.

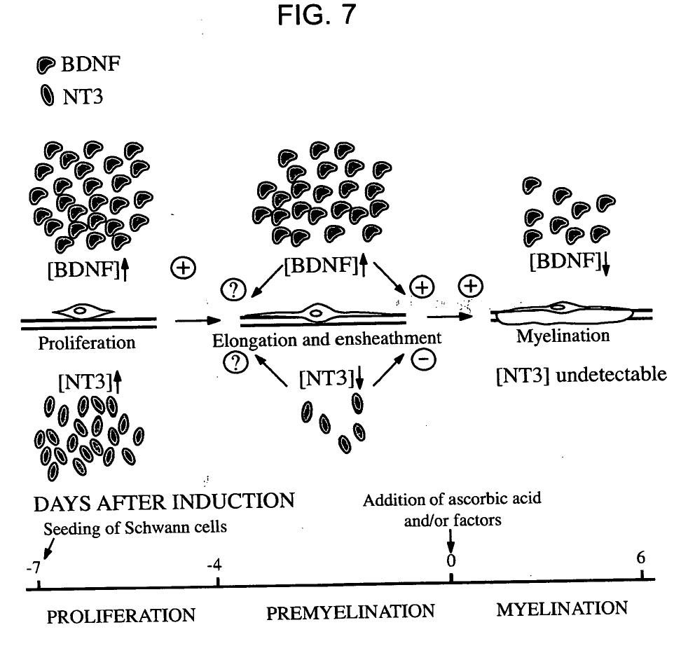

Similar analyses were also performed after injection with the chimeric protein TrkB-Fc (FIGS. 6A and B). By removing endogenous BDNF with the TrkB-Fc scavenger, a significant increase in the number of ensheathed axons (˜50%) was detected as compared with controls, and a corresponding decrease in the number of myelinated axons was also observed (FIG. 6C). In similar fashion, but with an opposite distribution as seen with the BDNF-treated nerves, TrkB-Fc had an effect not only in the proportion of axons that were myelinated, but also on the thickness of the remaining myelinated axons. In examining the distribution of the number of lamellae, the TrkB-Fc-treated nerves displayed thinner myelin sheaths or a greater population of axons with fewer wraps of myelin than the controls (FIG. 6D). These results were consistent with the Western blot analyses and with the effects observed in the cocultures, suggesting that BDNF plays a fundamental role in the initiation and progression of the myelination program of the peripheral nervous system.

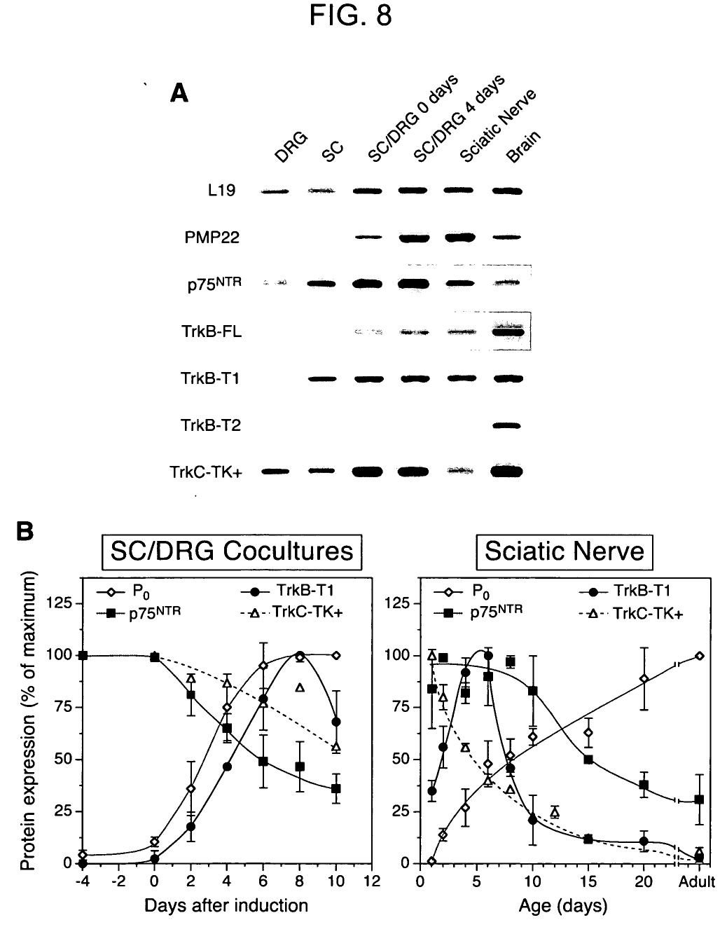

During development and/or after nerve injury, the complex interactions between glial cells and neurons are responsible for the reciprocal regulation and dramatic modulation of gene expression in both cell types. It is the action of multiple axonal/glial factors involved in an intricate neuron/Schwann cell cross-talk that allows for a fundamental relationship conducive to the formation of the myelin sheath. Schwann cell/neuronal cocultures provide a powerful tool to dissect the complex interactions necessary for the formation of peripheral myelin by permitting the characterization of three major stages in the process, proliferation, premyelination, and myelination stages. A schematic diagram of these stages including the expression of the neurotrophins is shown in FIG. 7.

The results described here show that the neurotrophins BDNF and NT3 are essential components in Schwann cell/neuronal interactions that prepare axons and_or Schwann cells for myelination. As determined by MAG and P0 synthesis, the active myelination stage begins in these cocultures by 2 days after induction with ascorbic acid. At this stage BDNF levels are significant but fall to undetectable levels 4 days after induction. That BDNF is indispensable for normal myelination is shown by the enhancement in the expression of the two myelin proteins both in the cocultures and in the sciatic nerve in vivo on addition of BDNF. This effect is further illustrated by the inhibition of myelin protein synthesis and the formation of myelin in cocultures when endogenous BDNF levels are reduced by addition of the receptor-based scavenger TrkB-Fc. The findings that BDNF increases the number of myelinating axons and the thickness of the myelin sheath in vivo add weight to the conclusion that BDNF is a key regulator of myelination and validate the coculture system as a reliable model for myelination.

In keeping with this are the further in vivo observations that TrkB-Fc increases the number of axons that remain in an ensheathed, premyelinating stage and reduces the number of myelin wraps of the remaining axons. BDNF is initially secreted by the DRG neurons and inhibition of its synthesis only occurs after induction of myelination.

The short-term (acute) modulation of neurotrophin levels was obtained by localized infusion of the factors or the scavengers in the area surrounding the sciatic nerve during development. This was done at a period in which the survival and innervation of the neurons had already been established. Therefore, the results obtained are the direct effect of the neurotrophins on the myelination process, and not an indirect effect due to the selection of a particular neuronal population. In agreement with this hypothesis, the ultrastructural studies did not reveal any differences in the total number of axons present in the BDNF- or TrkB-Fc-treated nerves compared with the contralateral control nerves. NT3 on the other hand inhibits myelination and in keeping with this the levels of NT3 secreted by cocultures decrease steadily over 5 days after seeding the Schwann cells until they are undetectable after induction. When NT3 is added at the day of induction, myelin protein synthesis in cocultures is decreased significantly and very few myelin internodes are observed. Addition of TrkC-Fc to remove endogenous NT3 increases myelin protein synthesis and restores normal myelination, again indicative of an inhibitory effect of NT3 on myelination. Of interest is the observation that NT3 applied in vivo does not inhibit myelin protein expression as much as it does in coculture. This difference may be due, in part, to a timing effect. Whereas NT3 was added to cocultures during the premyelination stage, in vivo injection took place postnatally and myelination may have already been initiated. This result also implies that NT3 exerts its inhibitory action during premyelination, a hypothesis consistent with the slight inhibition of protein synthesis observed 2 days after NT3 injection in vivo and not after 4 days. The limitations of in vivo studies might also be considered. Differences from animal to animal were always larger than between cocultures and although the contralateral leg was used as a control there is no simple way of discerning whether the lack of effect is due to the loss of NT3 responsiveness at center stage of myelin formation or to a decrease bioavailability of the factor. Nevertheless, BDNF and NT3 clearly have different modulatory effects at different stages in the myelination program.

Previous studies on the role of BDNF in nerve injury experiments have documented an increase in the amount of PNS myelin. Although these results confirm our own findings, it is difficult to exclude the influence of BDNF on the various stages preceding myelination, as well as its direct influence on the neuronal cells. In addition, studies have reported an increase in the expression of a Schwann cell myelin protein in cultures of quail nonneuronal cells in the presence of BDNF. Although the results of this study are quite similar to our data, the examination of mature myelin internodes was not explored. It seems evident from the results provided herein that BDNF is an essential component of the myelination program in the PNS.

The above data demonstrate that endogenous neurotrophins are key mediators of the myelination program in the PNS. The therapeutic implications of these findings relate specifically to the demyelinating neuropathies and to nerve injury. This previously uncharacterized role for neurotrophins on myelination will aid in the complex process remyelination. Neurons and Schwann cells share a mutual dependence in establishing or reestablishing a functional relationship through multiple axonal/glial signals. The mechanism of neurotrophin signaling is complex and depends on numerous factors. These signaling events rely heavily on the accessibility of the full-length and truncated Trk receptors and on the p75^{NTR}, the relative binding affinities and specificities of the ligands, and the relative amounts of the receptors and ligands present in the particular system. By elucidating the mechanism of neurotrophin action on the myelination process, and characterizing this previously uncharacterized neuronal/glial interaction, new therapeutic strategies into myelin repair and the functional recovery of demyelinating peripheral neuropathies is made possible.

### EXAMPLE 2

The neurotrophin receptor p75^{NTR} as a positive modulator of myelination.

Schwann cells in developing and regenerating peripheral nerves express elevated levels of the neurotrophin receptor p75^{NTR} Neurotrophins are key mediators of peripheral nervous system (PNS) myelination. The following results show that myelin formation is inhibited in the absence of functional p75^{NTR} and enhanced by blocking TrkC activity. Moreover, the enhancement of myelin formation by endogenous brain-derived neurotrophic factor (BDNF) is mediated by the p75^{NTR} receptor, while TrkC receptors are responsible for the neurotrophin-3 (NT3) inhibition. Thus p75^{NTR} and TrkC receptors have opposite effects on myelination.

The neurotrophin receptor p75^{NTR}(₁) is now known to have more diverse functions than that of being a helper for the Trk receptors. Here, we show that the neurotrophin BDNF acts through p75^{NTR} to enhance myelin formation. The neurotrophins, a family of growth factors including nerve growth factor (NGF), BDNF, NT3 and neurotrophin4/5 (NT4/5), exert their biological actions mostly in neuronal cells by regulating survival, differentiation and cell death(2). All known neurotrophins bind the receptor p75^{NTR}, but others of the Trk family of tyrosine kinase receptors are more selective about which neurotrophin they will bind. NGF binds to TrkA, BDNF and NT4/5 to TrkB, while NT3 binds to TrkC. Alternative splicing of the trkB and trkC genes results in full-length receptor isoforms (TrkB-FL and TrkC-TK+) containing an intact tyrosine kinase domain and the truncated isoforms (TrkB-T1 and -T2 and TrkC-TK−) that lack the kinase domain (3, 4).

The myelin sheath is a specialized membrane component in the nervous system that maximizes the efficiency and velocity of neuronal action potentials. The myelination program involves a number of signals between the neuronal and myelin forming cells that include, in the PNS, neuregulins(5), ATP(6), steroid hormones(7), Desert hedgehog(8), and the neurotrophins BDNF and NT3(9). Removal of BDNF inhibited myelination while removal of NT3 enhanced myelination in vitro and in vivo(9).

To identify the neurotrophin receptors responsible we determined which receptor mRNAs were present during myelination both in sciatic nerve and in Schwann cell/Dorsal Root Ganglia neuron (SC/DRG) cocultures by non-quantitative RT-PCR. The mRNAs for p75^{NTR} and TrkC-TK+ were present in both actively myelinating sciatic nerve and cocultures (FIG. 1A). TrkB-T1 mRNA was also detected in sciatic nerve and in cocultures while only a minute amount of TrkB-FL was observed. Myelination in the sciatic nerve, determined by the expression of the major myelin protein P₀, begins immediately after birth and continues over approximately 20 days. Myelination in cocultures occurs over 6 to 8 days after induction (FIG. 1B). The expression profile of the neurotrophin receptors was similar during myelination in the sciatic nerve and in coculture (FIG. 1). On the protein level, p75^{NTR} and TrkC-TK+ were present at high levels during myelination in both systems, decreasing only at later time points (FIG. 1B). TrkB-T1 protein levels correlated with active myelination, being induced at the initiation of myelination both in vitro and in vivo, reaching a peak at the time of maximum myelin accumulation and diminishing afterwards. TrkB-T1 expression may be an indicator of PNS myelination, while TrkB-FL protein levels in sciatic nerve during myelination were at least 100 times lower than that of TrkB-T1 (10, 11). p75^{NTR}, TrkB-T1 and full length TrkC receptors are, therefore, likely to be the major mediators of neurotrophin actions during PNS myelination.

The functions of p75^{NTR} and TrkB-T1 were analyzed by adding specific blocking antibodies in SC/DRG cocultures(12). Two different p75^{NTR} blocking antibodies [REX(13) and anti-p75] inhibited the accumulation of two major myelin proteins myelin-associated glycoprotein (MAG) and P₀ (FIG. 2A) and inhibited the formation of mature myelin internodes as shown by P₀ immunocytochemistry (FIG. 2C). In contrast, an antibody that blocks BDNF binding to TrkB(14) had the opposite effect, increasing myelin protein accumulation (FIG. 2A) and the number of mature myelin internodes (FIG. 2C). The blockade of all Trk-mediated tyrosine kinase activity by addition of K252a produced an increase in myelin protein accumulation (FIG. 2B) and mature myelin internode formation (FIG. 2C). This result is reminiscent of the effect obtained with the NT3 scavenger TrkC-Fc(9) and, most likely, could be attributed to the inhibition of TrkC activity. In the presence of Schwann cells, DRG neurons in culture become NGF-independent, and addition of K252a at the start of myelination does not affect neuronal or glial survival as determined by TUNEL analysis. In the presence of K252a, BDNF and TrkB-Fc are still able to modulate myelination (FIG. 2B), indicating that BDNF effects are not mediated by the tyrosine kinase activity of the TrkB-FL receptor. These results suggest that p75^{NTR} is the functional receptor that mediates the enhancement of myelination by endogenous BDNF, while TrkB-T1 acts in an inhibitory fashion, most likely by decreasing the availability of endogenous BDNF by competing with p75^{NTR} for its binding.

The function of p75^{NTR} was also analyzed during the development of the sciatic nerve in vivo. Subcutaneous injections of REX and/or BDNF were performed on newborn mice along the sciatic nerve(12). BDNF enhanced P₀ and MAG expression, whereas REX had an inhibitory effect (FIG. 2D). REX also blocked the enhancement achieved with BDNF. Electron microscopy analysis showed a decrease in myelin thickness in the sciatic nerves treated with REX when compared with the contralateral control nerves (FIG. 2E). Sciatic nerves from p75^{NTR} −/− mice(15) also displayed less than normal myelin thickness (FIG. 2F). While greater than 20% of the axons in control nerves had myelin sheaths with more than 30 wraps of myelin, very few axons presented such a degree of myelination if p75^{NTR} function was blocked either by REX treatment or by genetic deletion (p75^{NTR} −/− mice). Thus functional p75^{NTR} is necessary for proper myelination of the sciatic nerve during development. Sciatic nerves from adult p75^{NTR} −/− mice showed a large reduction in the number of myelinated axons (more than 50%), suggesting that the developmental decrease in myelination persists into adulthood. However, the selective decrease in specific neuronal populations in p75^{NTR} −/− mice complicates this analysis and a more thorough examination is still required.

The involvement of p75^{NTR} in the control of myelin formation by BDNF was further demonstrated in studies with p75^{NTR} −/− mutants, both in vivo and in vitro. Injection of BDNF along the sciatic nerves of wild-type mice enhanced P₀ and MAG protein expression (FIG. 3A-B). Likewise, removal of endogenous BDNF by injecting the neurotrophin scavenger TrkB-Fc resulted in the reduction of myelin protein expression. In contrast, neither BDNF nor TrkB-Fc were able to modulate myelin protein expression when injected in the p75^{NTR} −/− mice, in agreement with the premise that p75^{NTR} is the functional receptor for BDNF. The lack of BDNF activity was in sharp distinction with that of NT3. In both wild-type and p75^{NTR} −/− mutant mice, injection with NT3 inhibited and with TrkC-Fc enhanced myelination to the same degree. Similar conclusions were obtained using mouse SC/DRG cocultures(12). Myelin protein expression was enhanced by BDNF and decreased by TrkB-Fc in myelinating cocultures from wild-type embryos (FIG. 3C-D), while neither BDNF nor TrkB-Fc had any effect in cocultures from p75^{NTR} −/− embryos. Furthermore, NT3 inhibited and TrkC-Fc enhanced myelination both in wild-type and in p75^{NTR} −/− cocultures with the same efficiency, once again indicating that p75^{NTR} is the functional receptor for BDNF but not for NT3.

Our results demonstrate that neurotrophins are key mediators of PNS myelination and that different receptors are implicated in the positive and negative modulation by BDNF and NT3, respectively. A model illustrating their roles during myelination is depicted in FIG. 4. The binding of neurotrophins to p75^{NTR} and Trk receptors activate divergent intracellular pathways with Trk receptors preferentially activating pro-survival/mitogenic pathways(2). NT3 has been described as a pro-survival factor for SCs(16) and could, therefore, be acting like other ligands of tyrosine kinase receptors, such as neuregulins or FGF-2, by keeping the SCs in a proliferative, pre-myelinogenic state(5). On the other hand, less is known about the roles of p75^{NTR} and most of the studies have focused on its pro- and anti-apoptotic functions in neurons and the intracellular signaling pathways that are activated after NGF binding(2, 17). Although our results show that mature forms of neurotrophins modulate myelination, it may be possible that secreted proneurotrophins, that act as p75NTR-specific ligands(18), could also regulate myelination through p75^{NTR.} The complete ablation of all p75^{NTR} isoforms(19), including a splice variant that is unable to bind neurotrophins, produces a larger decrease in the number of neurons and SCs present in the sciatic nerve compared to the traditional p75^{NTR} −/− mice, suggesting an additional neurotrophin-independent role for this receptor. It remains unknown whether this is accompanied by a greater decrease in myelin. It is worth noting that the DRG neurons used in this study were maintained in NGF and the sensory fibers that grew and survived were NGF-dependent, which can constitute yet another layer of complexity in the interplay of neurotrophins and their receptors. Whether NGF and TrkA signaling contributes to myelination remains to be determined. Our results offer an example of how neurotrophins promote different effects according to whether p75^{NTR} or Trk is activated. Other instances in which such behavior has been documented include cell death/survival decisions in different neuronal types(2), and the differential regulation of neurotransmitter release by sympathetic neurons that produces a switch between excitatory and inhibitory neurotransmission(20).

An interesting characteristic of p75^{NTR} is its high level of expression in Schwann cells during development and in demyelination/remyelination paradigms(21). After nerve injury, the increase in p75^{NTR} expression is accompanied by an up-regulation of BDNF(22) and a decrease in NT3 expression (10). Aside from any effects on neuronal survival and axonal regrowth, these responses might also indicate a function in the myelination program. Our results indicating that p75^{NTR} regulates the myelination process in the PNS allows for the possibility of using specific p75^{NTR} agonists as therapeutic agents in instances in which increased myelination is required, such as peripheral neuropathies or nerve injury. Such compounds could mimic the pro-myelinating effects of BDNF without the undesired collateral consequences in the neuronal counterparts.

What is claimed is:

1. A method of treating a demyelinating condition, the method comprising:
administering an effective dose of one or both of (a) an agonist of the p75NTR receptor and (b) an antagonist or inhibitor of the Trk receptor to a patient suffering from said demyelinating conditions;
wherein the myelination of neurons is increased.

2. The method according to claim 1, wherein said neurons are peripheral neurons.

3. The method according to claim 2, wherein said condition is an injury to a nerve.

4. The method according to claim 2, wherein said condition is Marie-Charcot-Tooth disease.

5. The method according to claim 1, wherein said neurons are central nervous system neurons.

6. The method according to claim 5, wherein said condition is an injury to a nerve.

7. The method according to claim 5, wherein said condition is multiple sclerosis.

8. The method according to claim 5, wherein said condition is Guillain-Barré Syndrome.

9. A method of screening candidate agents for activity in enhancing myelination, the method comprising:
contacting a p75^{NTR} receptor with said candidate agent;
determining the ability of said agent to bind to, or act as an agonist of, said p75^{NTR};
wherein agonists of p75^{NTR} enhance myelination.

10. A method of screening candidate agents for activity in enhancing myelination, the method comprising:
contacting a Trk receptor with said candidate agent;
determining the ability of said agent to inhibits or antagonizes said Trk receptor;
wherein inhibitors or antagonists of Trk receptor enhance myelination.
